(19) United States
(12) Patent Application Publication
(10) Pub. No.: US 2006/0264896 A1
(43) Pub. Date: Nov. 23, 2006

(54) Minimally invasive apparatus and method for treatment of a tumor associated with a bone

(76) Inventor: Erika I. Palmer, Menlo Park, CA (US)

Correspondence Address: COOLEY GODWARD LLP;ATTN: PATENT GROUP, THE BOWEN BUILDING, 875 15TH STREET, N.W. SUITE 800, WASHINGTON, DC, 20005-2221, US

(21) Appl. No.: 11/124,387

(22) Filed: May 9, 2005

(51) Int. Cl. A61M 31/00

(52) U.S. Cl. 604/506; 604/164.01; 606/159; 600/562; 604/264

(57) ABSTRACT

A method includes percutaneously accessing a patient via a cannula, such that a distal end of the cannula is disposed substantially adjacent a tumor associated with a bone of the patient. A composition of the tumor is modified via a medical device movably disposed within the cannula. At least a portion of the tumor is removed through the cannula. The modifying is performed during a first time period and the removing is performed during a second time period. At least a portion of the first time period overlaps with at least a portion of the second time period.

### BACKGROUND

The invention relates generally to medical devices and procedures, and more particularly to a minimally invasive apparatus and method for use in the treatment of a tumor associated with a bone structure.

Some known methods of treating tumors associated with a bone structure involve invasive surgical techniques (i.e., surgical excision of the tumor). Radiation therapy is also used to treat tumors, which can be applied to the bone structure externally from or internally within the patient's body. Radiation therapy can also be applied following surgical excision of a tumor. Unfortunately, some tumors become resistant to radiation therapy, and a potential for damage to the spinal cord by the radiation exists.

Known techniques, such as the intratumoral injection of absolute alcohol or ethanol, have been developed to provide a non-invasive method of treating some tumors. Although these known techniques have reduced some of the risks associated with invasive surgical procedures and radiation therapy, a need for improvements still exists. For example, it may be desirable to avoid contact between the injected ethanol and healthy tissue near the treatment site of the tumor. Known methods of intratumoral injection do not provide a means for entirely preventing this contact. In addition, known methods of intratumoral injection do not provide for an effective method of removing the tumor after it has been treated with the ethanol.

Thus, a need exists for an apparatus and method for the minimally-invasive treatment of tumors associated with a bone structure, such as a vertebral body.

### SUMMARY OF THE INVENTION

A method includes percutaneously accessing a patient via a cannula, such that a distal end of the cannula is disposed substantially adjacent a tumor associated with a bone of the patient. A composition of the tumor is modified via a medical device movably disposed within the cannula. At least a portion of the tumor is removed through the cannula. The modifying is performed during a first time period and the removing is performed during a second time period. At least a portion of the first time period overlaps with at least a portion of the second time period.

### BRIEF DESCRIPTION OF THE DRAWINGS

The invention is described with reference to the accompanying drawings. In the drawings, like reference numbers indicate identical or functionally similar elements.

FIG. 1 is a schematic illustrating an apparatus according to an embodiment of the invention.

FIG. 2 is a plan view of the apparatus shown in FIG. 1 while in use to treat a tumor associated with a vertebral body.

FIGS. 3A through 3F are alternative sectional views taken along the line 3-3 in FIG. 2 according to various embodiments of the invention.

FIG. 4 is a side perspective view of an apparatus with a cut-away portion according to an embodiment of the invention.

FIG. 5 is a cross-sectional view taken along line 5-5 in FIG. 4.

FIG. 6 is a side perspective view of an apparatus according to an embodiment of the invention.

FIG. 7 is a side perspective view of an apparatus according to an embodiment of the invention.

FIG. 8 is a side perspective view of an apparatus according to an embodiment of the invention.

### DETAILED DESCRIPTION

The apparatus and methods described here allow percutaneous access to the internal or external area of a bone structure, via a cannula (or elongate body). A tumor associated with the bone structure can then be treated or its composition modified (e.g., dissolved or dislodged) with the use of medical devices configured to treat the tumor. Such medical devices can be configured to be movably disposed within a channel of the cannula. At least a portion of the treated or modified tumor can be removed from the body of a patient via the cannula. After the tumor has been removed from the body of the patient, the bone structure can receive further treatment, if necessary. For example, after removing a tumor from a vertebral body, a kyphoplasty procedure can be performed to restore the strength and/or height of the vertebral body, and/or alignment of the endplates. Although the following describes the use of the apparatus in spinal procedures, procedures on other areas of a body may be performed with the apparatus and methods described.

In one embodiment, a method includes percutaneously accessing a patient via a cannula, such that a distal end of the cannula is disposed substantially adjacent a tumor associated with a bone of the patient. A composition of the tumor is modified via a medical device movably disposed within the cannula. At least a portion of the tumor is removed through the cannula. The modifying is performed during a first time period and the removing is performed during a second time period. At least a portion of the first time period overlaps with at least a portion of the second time period.

In another embodiment, an apparatus includes a cannula having a proximal end portion and a distal end portion. The cannula is configured to percutaneously access a body of a patient such that the distal end portion of the cannula is disposed substantially adjacent a bone. A first medical device is configured to be received within the cannula. The first medical device is configured to modify a composition of a tumor associated with the bone. The cannula is further configured such that at least a portion of the tumor can be communicated from the distal end portion of the cannula to the proximal end portion of the cannula after the composition of the tumor has been modified by the first medical device.

In yet another embodiment, an apparatus includes an elongate body having a proximal end portion and a distal end portion. The elongate body is configured to percutaneously access a body of a patient such that the distal end portion of the elongate body is disposed adjacent to a tumor associated with the bone. The elongate body includes a first channel and a second channel. The first channel is configured to receive a medical device. The second channel is configured to communicate at least a portion of the tumor from within the body of the patient to a location outside of the body of the patient.

The term “medical device” is used here to mean a device that can be used in the treatment and/or modification of the composition of a tumor associated with a bone. For example, the medical device may provide a means of dissolving a tumor, dislodging a tumor, and/or removing a tumor associated with a bone structure.

The terms “cannula” and “elongate body” are used here to mean a component of the apparatus having one or more channels configured to receive a medical device therethrough and provide access to a tumor associated with a bone. For example, the cannula or elongate body can be substantially tubular. The cannula or elongate body can be a variety of different shapes and size, such as having a round or octagonal outer perimeter, and can include any suitable number of channels. In addition, the channel(s) can be a variety of different shapes and sizes, such as square, round, triangular, or any other suitable shape.

The term “a tumor associated with a bone” is used here to mean a tumor located at least partially in the interior of a bone, which is accessible through at least a portion of the bone, or a tumor located near the exterior of the bone, which is accessible from outside the bone. A tumor located in the interior of a bone can be accessed, for example, using known percutaneous accessing techniques, such as bone drilling.

Any of the embodiments or their components described herein can be constructed with any suitable material used for medical devices. For example, the cannula or elongate body, and the medical devices used in conjunction with the cannula or elongate body can each be constructed of stainless steel, shape memory alloys, titanium, polycarbonates, or other biocompatible materials.

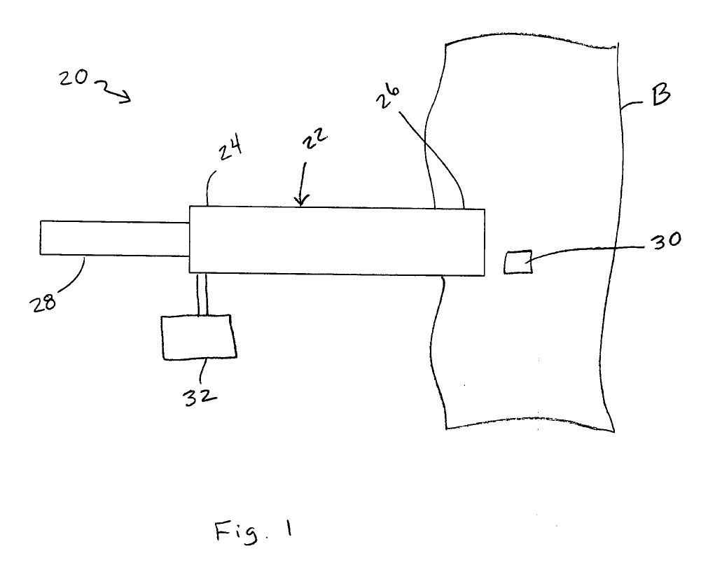

FIG. 1 is a schematic illustration of an apparatus 20 according to an embodiment of the invention. Apparatus 20 includes a cannula 22 having a proximal end portion 24, a distal end portion 26, and defining at least one channel (not shown in FIG. 1). The cannula 22 is configured to provide percutaneous-access to a bone structure B of a patient through the channel. The cannula 22 can be used to provide access to an interior or exterior portion of the bone structure, such as a vertebral body of the patient, to treat a tumor 30 associated with the bone structure.

The cannula 22 is configured to receive at least one medical device 28 that is movably disposed within the channel of the cannula 22. A variety of different types of medical device 28 can be used to perform various procedures on the tumor 30. For example, in one embodiment, the medical device 28 is an injection syringe used to modify the composition of the tumor through the injection of an agent, such as absolute ethanol. The medical device 28 (or alternatively a second medical device not shown in FIG. 1) can optionally also provide a means to remove at least a portion of the tumor 30 from the body of the patient after the tumor 30 has been treated or modified.

The apparatus 20 may also include a suction source 32 coupled to the cannula 22 to provide a suctioning force through a channel of the cannula 22. Due to the suctioning force, at least a portion of the modified or treated tumor is suctioned or removed from the body of the patient via the cannula 22 after the tumor has been treated or simultaneously when the tumor is treated. In alternative embodiments, the suction source 32 is coupled to a medical device 28 instead of the cannula 22.

In use, an access path in the patient's body can be created using known methods of percutaneously accessing a bone structure of a patient. Some known methods include the use of a bone drill or other similar device configured to penetrate a bone structure. For example, the flexible drill instrument disclosed in U.S. Patent Application 2003/0130664 can be used to create an access path. The disclosure of this application is incorporated herein by reference in its entirety. Such a device can be used in conjunction with apparatus 20.

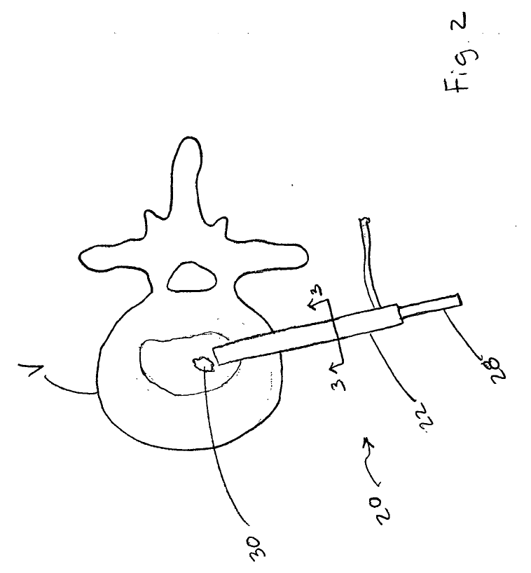

FIG. 2 illustrates the apparatus 20 used, for example, to treat a tumor associated with a vertebral body. The vertebral body can be accessed transpedicularly or extrapedicularly by using a guidewire inserted into the patient's body to plan a trajectory for a drill to follow. An access path can then be created using a bone drill (or other suitable device). The cannula 22 can be inserted into the access path and disposed substantially adjacent the tumor 30 associated with the bone structure, as illustrated in FIG. 2. The medical device 28 can be movably disposed within a channel of the cannula 22 to treat or modify the tumor 30. In some embodiments, the tumor 30 is modified during a first time period and at least a portion of the tumor 30 is removed during a second time period. At least a portion of the first time period can overlap with at least a portion of the second time period. Thus, the tumor 30 can be treated, while at the same time portions of the tumor 30 being treated can be removed through the cannula 22 via, for example, the suction source 32 (shown in FIG. 1) coupled to the cannula 22.

As stated above, in some embodiments, the suction source 32 is coupled to another device such as, for example, a second medical device (not shown in FIG. 1) insertable through a channel of the cannula 22. For example, a second medical device can be coupled to a suction source and can be movably disposed within a channel of the cannula. Such a medical device can be used in conjunction with the cannula 22 and also with a medical device 28 configured to treat a tumor. In other embodiments, a medical device 28 can remove at least a portion of the treated or modified (e.g., dislodged, dissolved) tumor from the body of the patient by means other than suctioning force as will be discussed in more detail below.

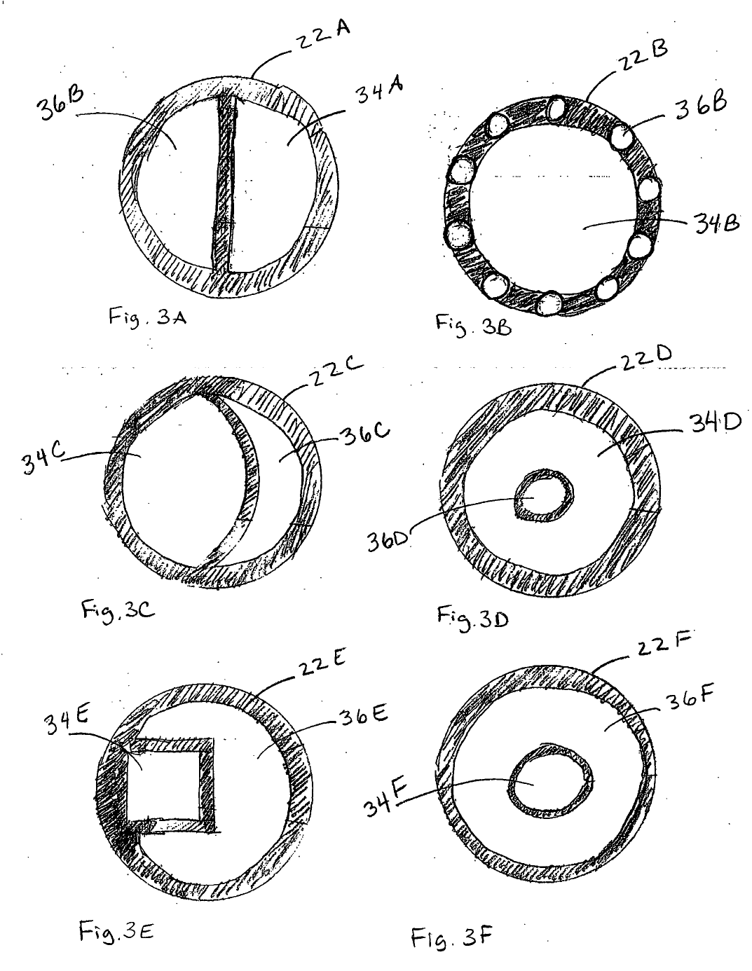

FIGS. 3A through 3F illustrate cross-sectional views of various examples of embodiments of the cannula 22 of the invention. As shown in FIGS. 3A through 3F, the cannula 22 can include at least one channel 34 and at least one channel 36. In some embodiments, channel 34 can be configured to receive a medical device 28 and channel 36 can be configured to communicate at least a portion of the treated or modified tumor from a location within the body of the patient to a location outside the body of the patient via a suctioning force produced by a suction source as described above.

The illustrated embodiments shown in FIGS. 3A through 3F are merely examples of possible configurations of cannula 22 and are not intended to be exhaustive. Cannula 22 can include a variety of different shapes and sizes, and channels 34 and 36 can be a variety of different shapes and sizes. In addition, the cannula 22 may be configured with more or less than two channels. For example, FIG. 3B illustrates a cannula 22B having a channel 34B configured to receive a medical device 28, and multiple channels 36B configured to communicate the treated tumor through the cannula 22 via a suctioning force. Also, the functions of the channels for a particular configuration of a cannula 22 may be reversed. For example, FIGS. 3D and 3F illustrate cannula 22D and cannula 22F, respectively, having substantially identical cross-sections, (i.e., a smaller circular channel and a larger radially extended channel). The cannula 22D includes a larger radially extending channel 34D configured to receive a medical device 28, and a smaller circular channel 36D configured to remove a portion of the treated tumor via a suctioning force. The cannula 22F includes a smaller circular channel 34F configured to receive a medical device 28, and a larger radially extending channel 36F configured to remove a portion of the treated tumor via a suctioning force.

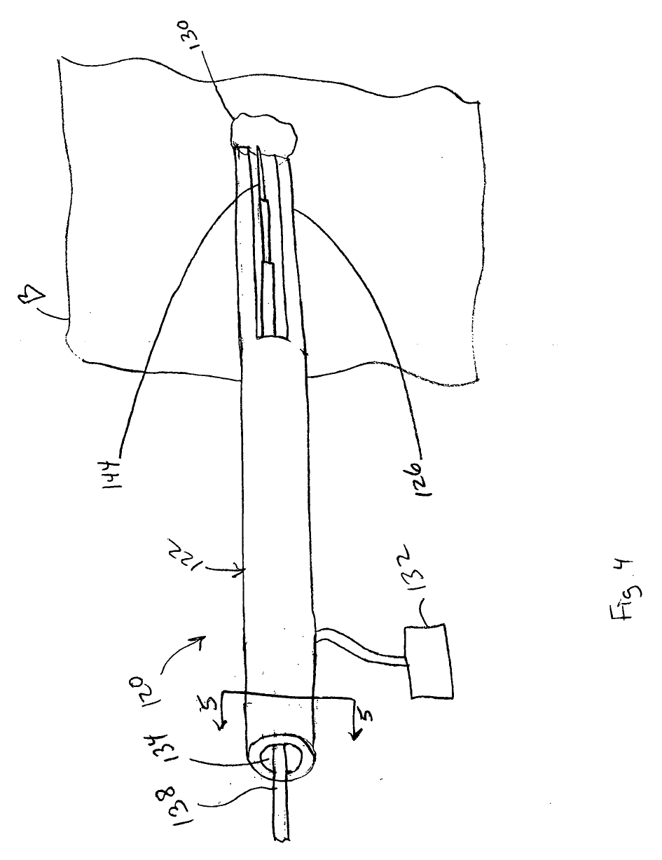

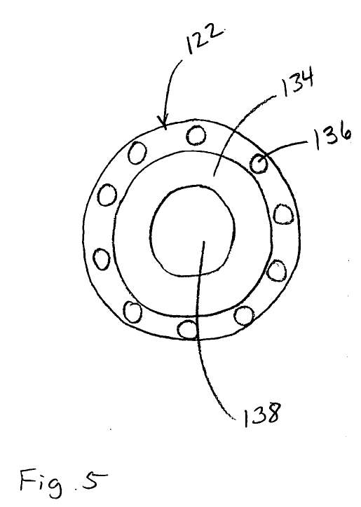

The embodiments illustrated in FIGS. 3A, 3C and 3E are examples of other possible shapes and sizes of the channels 34 and 36. For example, FIG. 3A illustrates a cannula 22A having two channels that are substantially equal in size and shape. FIG. 3C illustrates a cannula 22C having a larger channel 34C than channel 36C. FIG. 3E illustrates an embodiment of a cannula 22E having a square shaped channel 34E and a substantially semi-circular channel 36E. As stated above, the functions of the channels can also be reversed. [10321 Several additional exemplary embodiments are now described. These embodiments are only exemplary, and many other combinations of the cannula and medical device(s) are possible. FIGS. 4 and 5 illustrate an apparatus 120 according to an embodiment of the invention. Apparatus 120 includes a cannula 122 having a first channel 134, multiple suctioning channels 136 (see FIG. 5), and a medical device 138. First channel 134 is configured to receive a medical device, such as medical device 138. The multiple suctioning channels 136 are coupled to a suction source 132. Medical device 138 includes a fine gage needle 144 and can be configured to inject an agent into a tumor 130. The agent can be, for example, absolute ethanol (EtoH), chymopapain, steroids (prednisone, and dexamethasone) or any other agent suitable for the treatment of tumors. Such an agent can be used to cause the tumor 130 to at least partially dissolve, or otherwise modify the composition of the tumor 130.

In use, the cannula 122 can be positioned such that a distal end portion 126 of cannula 122 is contacting tumor 130 associated with a bone structure B, as shown in FIG. 4. The medical device 138 can be movably disposed within the channel 134 of cannula 122 such that the needle 144 contacts the tumor 130, as shown in the cut-away portion in FIG. 4. An agent can be injected into the tumor 130 via the medical device 138, while at the same time a suctioning force can be applied to the channels 136 of the cannula 122. The positioning of the cannula 122 with the distal end portion 126 contacting the tumor site, allows any overflow of the injected agent to be contained within a perimeter of the cannula 122. In addition, the simultaneous suctioning force applied from within the perimeter of the cannula 122 enables dissolved portions of the tumor 130, as well as the residue agent, to be timely removed from the patient's body via the cannula 122 in a minimally-invasive manner. Thus, the agent can be injected into the tumor 130 and removed from the patient's body via medical device 138, while minimizing any adverse effects to the healthy tissue and bone surrounding the tumor treatment site.

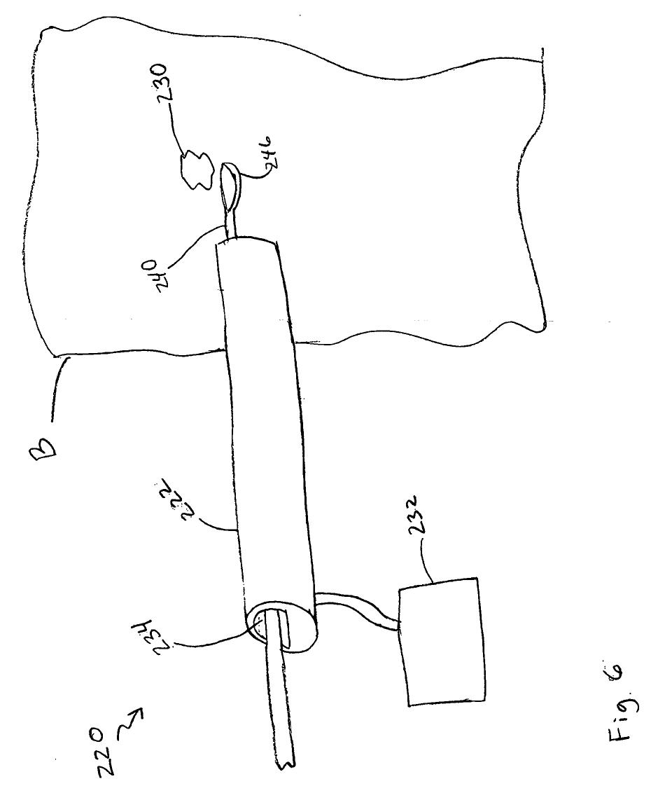

An apparatus 220 according to another embodiment of the invention is illustrated in FIG. 6. Apparatus 220 includes a cannula 222 having a channel 234 that receives a medical device 240 and a second channel (not shown) coupled to a suction source 232. The medical device 240 includes a scoop portion 246. The medical device 240 is movably disposed within the channel 234 of cannula 222 such that the scoop portion 246 is positioned adjacent a tumor 230 associated with a bone structure B. Scoop portion 246 is configured to dissect or dislodge at least a portion of the tumor 230 from bone structure B. The portion of the dislodged tumor 230 can then be removed (e.g., suctioned) from the body of the patient through the second channel of the cannula 222.

In alternative embodiments, a medical device can also be used to remove portions of the dislodged tumor instead of, or in addition to, using the suctioning capability of the cannula. For example, a scoop portion of the medical device can be configured to capture the dislodged portion of the tumor and then the medical device can be removed from the cannula (with the dislodged portion of the tumor captured within the scoop portion).

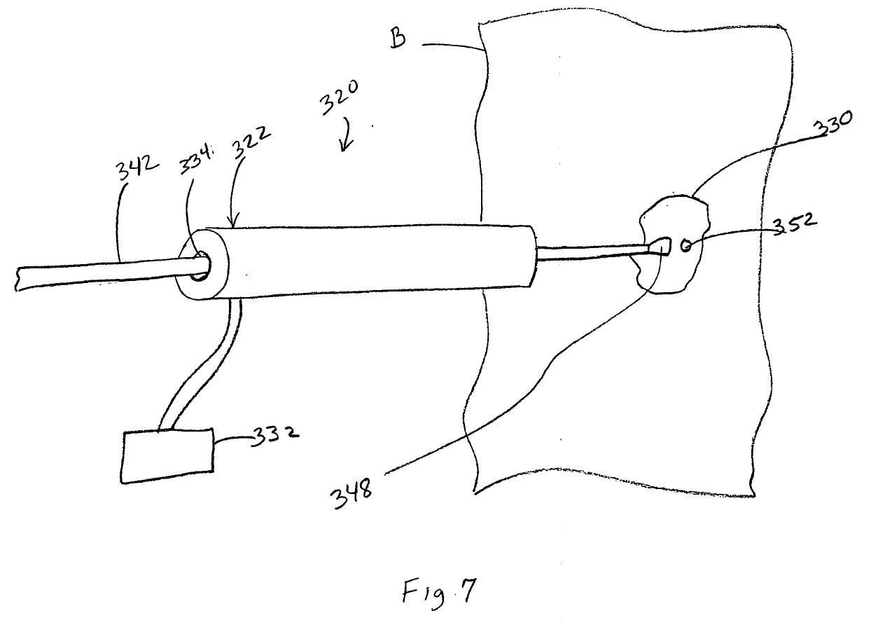

FIG. 7 illustrates an apparatus 320 according to another embodiment of the invention. Apparatus 320 includes a cannula 322 having a channel 334 that receives a medical device and a second channel (not shown) coupled to a suction source 332. A medical device 342 is movably disposed within channel 334, and the distal end of medical device 342 can be positioned adjacent a tumor 330 associated with a bone structure B. The medical device 342 includes a probe 348 configured to inject or deposit one or more markers 352 into the tumor 330, thereby changing the composition of the tumor 330. The marker 352 can be, for example, a metal bead. The marker 352 can be used, for example, by a radiation technician to calculate the exact position of the tumor 330 to aid in the delivery of a precise dose of radiation to the tumor 330. In some embodiments, three markers 352 are deposited into a tumor to allow the technician to triangulate the exact position of the tumor. In conjunction with an imaging procedure with a device such as an x-ray machine or a virtual imaging modality (e.g., computed tomography (CT), magnetic resonance imaging (MRI)), the marker 352 can allow for the location of the tumor 330 to be determined externally to the body of the patient and prior to removing the tumor 330 from the body of the patient. In an alternative use, after the tumor has been removed, radioactive beads can be delivered to the tumor's prior location within the patient's body to irradiate any possibly remaining cancerous cells. For radioactive beads having a relatively short half-life, the beads can be left in the patient's body; for radioactive beads having a relatively longer half-life, the beads can be removed at a later time.

In other alternative uses, after the location of the tumor 330 has been identified, other medical devices, such as medical device 138 and/or medical device 240, can be used to further treat or modify the composition of the tumor as described above. The treated portions of the tumor can be suctioned through the second channel of the cannula 322 and out of the body of the patient simultaneously with the modifying of the tumor 330 as previously described. In yet another alternative use, after the location of the tumor has been identified, a biopsy sample of the tumor can be taken.

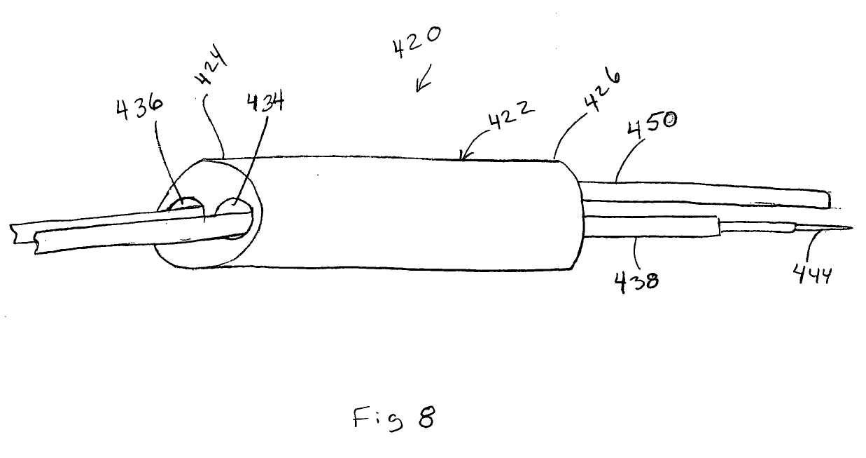

FIG. 8 illustrates an apparatus 420 according to another embodiment of the invention. Apparatus 420 includes a cannula 422 having a proximal end portion 424 and a distal end portion 426. The cannula 422 defines a first channel 434 and a second channel 436. Channel 434 can receive a first medical device, such as medical device 438 shown in FIG. 8. Channel 436 can receive a second medical device 450. The channels 434 and 436 can be any variety of different shapes and sizes. For example, the cannula 422 can have a cross-section as shown for cannula 22 in FIGS. 3A and 3C-3F. In some embodiments, the cross-sectional shapes of the channels 434 and 436 match the cross-sectional shapes of the medical devices movably disposed in the channels 434 and 436. In addition, cannula 422 can include more or less than two channels.

The first medical device 438, as shown, includes a needle 444 configured to penetrate a tumor associated with a bone. Although medical device 438 is shown, it should be understood that medical device 438 is shown for illustrative purposes only and that any other suitable medical device can be used with cannula 422. The second medical device 450 can be, for example, a medical device configured to provide suctioning force. For example, second medical device 450 can be coupled to a suction source instead of and/or in conjunction with cannula 422.

In use, the first medical device 438 can be inserted through channel 434 such that the needle 444 contacts a tumor (see also FIG. 4). The first medical device 438 can modify the composition of the tumor, for example, by injecting an agent into the tumor causing the tumor to at least partially dissolve. The second medical device 450 can be inserted through the channel 436 and positioned adjacent the tumor. The second medical device 450 can use its suctioning force to communicate at least a portion of the dissolved tumor from a location within the body of the patient to a location outside the body of the patient. Thus, in this embodiment, the cannula 422 does not directly communicate a suctioning force to a location near the tumor. As described above, the treatment of the tumor with medical device 438 can occur simultaneously with the suctioning of the treated or dissolved portions of the tumor through the cannula 422, via the second medical device 450.

In an alternative embodiment, the cannula can include a sharpened distal end portion configured to penetrate the bone structure. Thus, access to the bone structure can be made with the cannula, rather than using a separate device such as a bone drill. In such an embodiment, a medical device configured as a stylet can be used in conjunction with the cannula. The stylet can be, for example, configured with an outer perimeter that matingly fits within a channel of the cannula. The stylet can be positioned within the channel of the cannula when the cannula is used to penetrate the bone structure. The stylet can prevent bone material from pushing into the channel of the cannula when the cannula is penetrating the bone structure. The stylet can then be removed from the channel once the cannula has accessed the desired location, e.g., the interior of the bone structure, and a different medical device can be disposed within the channel to perform a medical procedure on a tumor associated with the bone structure as described above.

### Conclusion

While various embodiments of the invention have been described above, it should be understood that they have been presented by way of example only, and not limitation. The invention has been particularly shown and described with reference to specific embodiments thereof, but it will be understood that various changes in form and details may be made.

For example, a variety of different medical devices can be used in conjunction with the cannula of the invention, and more than two medical devices can be used. For example, a medical device configured to inject a marker into the tumor can be used together with both a medical device configured to dissolve or dislodge a tumor and a medical device configured to remove a portion of the modified tumor from the body of the patient.

What is claimed is:

1. A method, comprising:
percutaneously accessing a patient via a cannula such that a distal end of the cannula is disposed substantially adjacent a tumor associated with a bone of the patient;
modifying a composition of the tumor via a medical device movably disposed within the cannula; and
removing at least a portion of the tumor through the cannula,
the modifying being performed during a first time period,
the removing being performed during a second time period, at least a portion of the first time period overlaps with at least a portion of the second time period.

2. The method of claim 1, wherein the percutaneously accessing includes percutaneously accessing an interior of the bone, the modifying and the removing being performed on the tumor disposed within the interior of the bone.

3. The method of claim 1, wherein the removing includes suctioning the portion of the tumor through the cannula.

4. The method of claim 1, wherein the removing includes inserting into the cannula a second medical device configured to provide a suctioning force to remove the portion of the tumor.

5. The method of claim 1, wherein:
the modifying includes dissolving at least a portion of the tumor with an agent.

6. The method of claim 1, wherein:
the modifying includes dissolving at least a portion of the tumor with an agent, the agent being at least one from the group of an ethanol and a chymopapain.

7. The method of claim 1, wherein the modifying includes injecting the tumor with a marker via the medical device, the method further comprising:
determining a location of the tumor within the body of the patient based on a position of the marker, the determining being performed externally from the patient and before the removing.

8. The method of claim 1, wherein:
the modifying being performed through a first channel of the cannula; and
the removing being performed through a second channel of the cannula different from the first channel of the cannula.

9. An apparatus, comprising:
a cannula including a proximal end portion and a distal end portion, the cannula configured to percutaneously access a body of a patient such that the distal end portion of the cannula is disposed substantially adjacent a bone; and
a first medical device configured to be received within the cannula, the first medical device configured to modify a composition of a tumor associated with the bone,
the cannula being further configured such that at least a portion of the tumor can be communicated from the distal end portion of the cannula to the proximal end portion of the cannula after the composition of the tumor has been modified by the first medical device.

10. The apparatus of claim 9, wherein:
the first medical device is configured to modify the composition of the tumor during a first time period,
the cannula is configured such that at least the portion of the tumor is communicated during a second time period, and
at least a portion of the first time period overlaps with at least a portion of the second time period.

11. The apparatus of claim 9, wherein:
the first medical device includes a scoop portion, the scoop portion configured to dislodge the portion of the tumor from the bone.

12. The apparatus of claim 9, wherein:
the first medical device includes a probe, the probe configured to inject into the tumor a marker configured to identify a location of the tumor within the body of the patient.

13. The apparatus of claim 9, wherein:
the first medical device including a needle, the needle configured to inject into the tumor an agent configured to dissolve the portion of the tumor.

14. The apparatus of claim 9, further comprising:
a second medical device configured to be received within the cannula, the second medical device configured to provide a suctioning force such that at least a portion of the tumor is removed from the body of the patient.

15. An apparatus, comprising:
an elongate body, the elongate body including a proximal end portion and a distal end portion, the elongate body configured to percutaneously access a body of a patient such that the distal end portion of the elongate body is disposed adjacent to a tumor associated with the bone, the elongate body including a first channel and a second channel, the first channel configured to receive a medical device, the second channel configured to communicate at least a portion of the tumor from within the body of the patient to a location outside of the body of the patient.

16. The apparatus of claim 15, wherein:
the first channel configured to receive the medical during a first time period,
the second channel configured to communicate at least the portion of the tumor from within the body of the patient to the location outside of the body of the patient during a second time period,
at least a portion of the first time period overlapping with at least a portion of the second time period.

17. The apparatus of claim 15, further comprising:
the medical device, the medical device including a scoop portion, the scoop portion configured to dislodge at least the portion of the tumor from the bone, the second channel configured to communicate the dislodged portion of the tumor from within the body of the patient to a location outside of the body of the patient.

18. The apparatus of claim 15, further comprising:
the medical device, the medical device including a needle, the needle configured to inject into the tumor an agent configured to dissolve at least the portion of the tumor, the second channel configured to communicate the dissolved portion of the tumor from within the body of the patient to a location outside of the body of the patient.

19. The apparatus of claim 15, further comprising:
the medical device, the medical device being a probe, the probe configured to inject into the tumor a marker configured to identify a location of the tumor within the body of the patient.

20. The apparatus of claim 15, further comprising:
the medical device, the medical device being a first medical device; and
a second medical device different from the first medical device, the first channel configured to receive the first medical device, the second channel configured to receive the second medical device, the second medical device configured to remove at least the portion of the tumor from within the body of the patient to a location outside of the body of the patient.

21. The apparatus of claim 15, further comprising:
the medical device, the medical device being a first medical device; and
a second medical device different from the first medical device, the first channel configured to receive the first medical device, the second channel configured to receive the second medical device, the second medical device configured to remove at least the portion of the tumor from within the body of the patient to a location outside of the body of the patient, the second medical device configured to provide a suctioning force.
